(19) United States
(12) Patent Application Publication
(10) Pub. No.: US 2019/0292115 A1
(43) Pub. Date: Sep. 26, 2019

(54) HOMOGENEOUS MICROORGANISM EXTRACT USING USEFUL AND FUNCTIONAL MICROORGANISMS AND METHOD FOR PRODUCING SAME

(71) Applicant: Yoon Soo Choi, Seoul (KR)

(72) Inventor: Yoon Soo Choi, Seoul (KR)

(21) Appl. No.: 15/506,752

(22) PCT Filed: Aug. 31, 2015
(86) PCT No.: PCT/KR2015/009147

§ 371 Date: Jul. 16, 2018

(30) Foreign Application Priority Data
Sep. 1, 2014 (KR) 1020140115648

(51) Int. Cl. C05F 11/08; C12N 1/20; C12N 1/16; C05F 17/00

(52) U.S. Cl. CPC C05F 11/08; C12N 1/20; C05F 17/009; C05F 17/0018; C12N 1/16

(57) ABSTRACT

The present invention pertains to a homogeneous microorganism extract using useful and functional microorganisms and a method for producing the same, and particularly to: a homogeneous microorganism extract which is obtained by homogenizing and extracting a complex enzyme, various inducing substances and the like by using useful microorganisms and functional microorganisms that are isolated from soil for the purpose of soil improvement, crop growth and pest control; and a method for producing said homogeneous microorganism extract. The method is technically characterized by the following: the homogeneous microorganism extract using useful and functional microorganisms and the method for producing the same are characterized in that: microorganisms are homogenized with a complex enzyme (protein, sugars, fat, fiber, synthetic enzymes for acids and alkalis, and decomposition enzymes etc), primary metabolites, secondary metabolites, antibiotic substances, various active materials, and the like, which are produced through combination culture in which cultured useful microorganisms and functional microorganisms are made and cultured under a symbiotic relationship such that the useful microorganisms and functional microorganisms serve as a substrate and a raw material; then microorganism inducing substances, crop growth inducing substances, and pest resistance inducing substances are extracted from microorganism cells; and the resultant extract is sterilized and concentrated. Therefore, since said extract includes the complex enzyme, various inducing substances, and the like, the functions of soil improvement, crop growth and pest control are achieved. Accordingly, the present invention is advantageous in cost saving and income increase; advantageous in that the types and number of useful microorganisms can be increased by inducing sunflower bacteria that has been already settled, instead of settling the microorganisms on soil and crops as in conventional microorganism formulations; and advantageous in that the extract directly affects crops so that desired effects are relatively quickly expressed and microbial flora is improved.

### BACKGROUND

Today, current farming destroyed the ecosystem as well as human because of contamination of soil, water quality and agricultural products due to the overdose of pesticide and chemical fertilizer. So the solution to such problems is eco-friendly organic agriculture.

Organic agriculture natural farming that maintains material circular systems and symbiotic relationships between human and organisms in nature by using natural materials only, not including synthetic chemical materials

Microorganisms agricultural materials among lots of organic agricultural materials to doing organic farming are not generalized but commercialized.

Microorganisms agricultural materials' are agricultural material that sprayed soil and plant directly, because of agricultural product quality improvement, increasing yield amount, soil amelioration, pest control and eliminating contaminants.

Conventional patent, ‘eco-friendly organic liquid fertilizer and method thereof’ (application No. 10-2012-0048556), is an example of microorganisms agricultural materials.

Followed conventional patent, organic liquid fertilizer consisted of different ingredient including all of organic and inorganic materials by fermenting after mixing difference ratio both nitrogen source and carbon source each other, has a feature of using by-products made by recycling agricultural, animal husbandry and fisheries.

Specifically, conventional patent induce a difference ratio of fermenting liquid ingredient by fermenting after mixing difference ratio both nitrogen source and carbon source each other and produce a difference ratio liquid fertilizer by using obtained a difference ratio of fermenting liquid standard. Organic liquid fertilizer, consist of obtained fermenting liquid that is used fertilizer by itself or mixing other fermenting liquid to have various fertilizer ingredient before using, is produced by eco-friendly methods.

Thus, current microorganism agricultural materials are almost living microorganism materials. So these have lots of troubles including supplying functionality production of settled living microorganisms to soil and plants, competence of native microorganisms, temperature, soil feature (pH, EC etc.) optimized for living microorganisms.

### SUMMARY

Microorganism extract using the effective microorganisms and functionality microorganism and the production method thereof is produced complex enzyme and various inducer and microorganism extract by using effective microorganisms and functionality microorganisms isolated all in the soil for and the production method. The products of microorganism homogeneous extract that is extracting both incubated effective microorganisms and functionality microorganisms incubating sole and mixing each other because of producing lots of complex enzyme(proteins, sugars, fatty acids, cellulose, acid and alkali synthetic enzyme and decomposition enzyme etc.), primary metabolite, secondary metabolite, antibiotics, various active materials to form new microorganisms ecological systems including subtle and complex symbiotic relationship and microorganism inducer, plant growth promoter, plant pest resistance inducer etc. when homogenizing microorganisms, microorganism extract are sterilized and concentrated.; is a feature and the methods of thereof is a technical feature. This invention has an effect of plant growth promoting, plant pest control and soil ameliorate including complex enzymes and various inducing materials, and so has an effect of decreasing product cost, increasing in income. And this invention does not immigrant microorganisms into soil and plants like a conventional microorganism material but induces in situ neutral microorganisms settled already. So, it has an effect of increasing types and population of effective microorganisms and relatively fast improvement microorganism system.

### BRIEF DESCRIPTION OF THE DRAWINGS

FIG. 1 is a schematic configuration diagram showing a microorganism extract using the effective microorganisms and functionality microorganism and the production method.

FIG. 2 is a schematic configuration diagram showing an example of a culturing tank followed a microorganism extract using the effective microorganisms and functionality microorganism and the production method.

FIG. 3 is a schematic configuration diagram showing a picture of soil microorganisms isolation followed a microorganism extract using the effective microorganisms and functionality microorganism and the production method.

FIG. 4 is a schematic configuration diagram showing a pepper growth rate picture of a comparison of sprayed a microorganism extract using the effective microorganisms and functionality microorganism and non-sprayed.

FIG. 5 is a schematic configuration diagram showing a pepper anthrax resistance picture of a comparison of sprayed a microorganism extract using the effective microorganisms and functionality microorganism and non-sprayed.

FIG. 6 is a schematic configuration diagram showing a rice growth rate picture of a comparison of sprayed a microorganism extract using the effective microorganisms and functionality microorganism and non-sprayed.

FIG. 7 is a schematic configuration diagram showing a picture of soil physics improvement sprayed a microorganism extract using the effective microorganisms and functionality microorganism after 3 months.

### DETAILED DESCRIPTION

### Technical Task

This invention is created due to solve problem of the conventional problems. Effective microorganisms and functionality microorganisms isolated all in the soil cultivate by sole and mixing due to ameliorate the soil, plant growth and pest control. Incubated effective microorganisms and functionality microorganisms incubate sole and mixing each other because of producing lots of objective materials using to form new microorganisms ecological systems including subtle and complex symbiotic relationship. Both complex enzyme, primary metabolite, secondary metabolite, antibiotics, various active materials etc. when combination incubating and microorganism inducer, plant growth promoter, plant pest resistance inducer etc. by homogenizing microorganisms are extracted, thus after extracting, microorganism extract are sterilized and concentrated. It is an object of the present invention to provide microorganism extract and method thereof.

It is also an object of the present invention to provide microorganism extract enhanced functionality for achieving various purposes and the method thereof.

It is also an object of the present invention to provide microorganism extract in which is not immigrant microorganisms into soil and plants like a conventional microorganism material but induce in situ neutral microorganisms settled already, and so, it has an effect of increasing types and population of effective microorganisms and relatively fast improvement microorganism system and method thereof.

It is, therefore, an object of the present invention to provide the incubation technique creating various object substance made by effective microorganisms and functionality microorganisms, the technique of homogenizing and extracting various object substance, providing biological control agents applying superior antagonistic microorganisms and crop growth agent applying effective microorganisms and the producing method of soil ameliorant in which applied various enzyme complex, metabolite and inducer produced by effective microorganisms.

Other objects, features and advantages of the present invention will become apparent from the following detailed description of the present invention when taken in conjunction with the accompanying drawing.

### Task Solution

For achieving following task solution that present invention want to solve, the present invention has a technical feature like followed.

Effective microorganisms and functionality microorganisms isolated all in the soil cultivate by sole and mixing due to ameliorate the soil, plant growth and pest control. The products of microorganism homogeneous extract that is extracting both incubated effective microorganisms and functionality microorganisms incubating sole and mixing each other because of producing lots of complex enzyme(proteins, sugars, fatty acids, cellulose, acid and alkali synthetic enzyme and decomposition enzyme etc.), primary metabolite, secondary metabolite, antibiotics, various active materials to form new microorganisms ecological systems including subtle and complex symbiotic relationship and microorganism inducer, plant growth promoter, plant pest resistance inducer etc. by homogenizing microorganisms, microorganism extract are sterilized and concentrated.; is a feature.

At this time, effective microorganisms isolated in soil including Bacillus sp., lactic acid bacteria genus, Saccharomyces sp. Actinomyces sp. and photosynthesis bacteria genus; is a technical feature.

At this time, microorganisms isolated soil including Azotobacter sp., Pseudomonas sp., Aspergillus sp., Penicillium sp., Clostridium sp. and Trichoderma sp.; is a feature.

Also, producing method of the microorganism extract included following steps, preparatory stage including measuring raw material using incubation, eliminating impurities, sterilizing raw material using ultra high temperature; object material producing stage including inoculating anyone or any of several functionality microorganisms, anyone or any of several effective microorganisms in previous preparatory stage material and incubating 20° C.˜35° C., 35° C.˜60° C. for 3˜5 days, separately; separating stage including separating object material produced during previous object material producing stage and media in incubating tanks, separately; combination incubating stage including mixing object material separated previous separating stage depending on the blending ration according to purpose and incubating with culturing raw material for 5˜7 days, separately; separating stage including separating combination incubating material produced during previous combination incubating stage and raw material, separately; stirring stage including stirring each combination incubating material separated during previous separating stage according to purpose; sterilizing stage including sterilizing microorganisms in each combination incubating material stirred during previous stirring stage by using ultraviolet ray; microorganism homogeneous stage including homogenizing microorganism cell sterilized during previous sterilizing stage by using high pressure; cell wall fragment separating stage including separating homogeneous material and homogeneous cell wall fragment made during previous microorganism homogeneous stage; concentrating stage including concentrating homogeneous material without homogeneous cell wall fragment separated during cell wall fragment separating stage; is a technical feature.

Also, preparatory stage including sterilizing with 6 kg/cm² high temperature autoclaved; is a technical feature.

Also, stirring stage including stirring combination incubating material with 200˜250 rpm/min in stirring tanks for 3 days; is a technical feature.

Also, sterilizing stage including sterilizing combination incubating material by using 30,000 μW·lm/m³ ultraviolet ray; is a technical feature.

Also, microorganism homogeneous stage including homogenizing microorganism cell by using 2,000 bar/cm² pressure; is a technical feature.

Also, cell wall fragment separating stage including separating homogeneous material and homogeneous cell wall fragment by using 40˜50 kg/cm² centrifugal force; is a technical feature.

Also, concentrating stage including concentrating homogeneous material with 1.2˜1.5 specific gravity under 60° C.; is a technical feature.

### Effect of Invention

Through the followed task solution, microorganism extract including complex enzyme, primary metabolite, secondary metabolite, antibiotics, various active material and various inducer has an effect of plant growth promoting, plant pest control and soil ameliorate including complex enzymes and various inducing materials, and so has an effect of decreasing product cost, increasing in income.

The method of microorganism extract to produce following present invention has an effect that enhanced functionality for achieving various purposes according to purposes.

Also, the present invention has an effect that microorganism extract in which is not immigrant microorganisms into soil and plants like a conventional microorganism material but induce in situ neutral microorganisms settled already, and so, it has an effect of increasing types and population of effective microorganisms and relatively fast improvement microorganism system.

So, it has an effect of plant pest control and soil amelioration as well as increasing agricultural output and quality.

Also, The present invention is not influenced by soil environment factor including temperature and soil texture than conventional probiotic materials due to treated sterilization after homogenizing microorganisms and extracting various object material produced by microorganisms and has features including easy handling and long-term storage possible.

### Best Mode for Carrying Out the Invention

The following detailed description of the invention refers to the accompanying drawings, which illustrate, by way of example, specific embodiments in which the invention may be practiced. These embodiments are described in sufficient detail to enable those skilled in the art to practice the invention. It should be understood that the various embodiments of the present invention are different, but need not be mutually exclusive. For example, the specific shapes, structures, and characteristics described herein may be embodied in this embodiment. It is also to be understood that the position or arrangement of the individual components within each disclosed embodiment may be varied without departing from the spirit and scope of the present invention. The following detailed description is, therefore, not to be taken in a limiting sense, and the scope of the invention is to be limited only by the appended claims, along with the full scope of equivalents to which such claims are entitled. In the drawing, like reference numerals refer to the same or similar functions throughout the several views.

The present invention makes microorganism extract through the technical feature like followed.

Various object material are extracted by incubating sole and mixing each other to form new microorganisms ecological systems including subtle and complex symbiotic relationship.

Also, various object material are extracted by mixing aerobic, anaerobic and semi-aerobic microorganism to form various effective and functionality microorganisms, after incubating.

Also, various object material are extracted by mixing effective microorganisms and functionality microorganisms for one population which made a functional material with a superior function compared with general microorganisms, after incubating.

Also, various object material extracted by controlling conditions for optimal growth of microorganisms.

Also, functional material that is not made sole incubation is extracted by mixing microorganisms each other including competition or symbiosis relationships.

Also, functional material that is not made sole incubation is extracted by mixing microorganisms each other including aerobic microorganisms and anaerobic microorganisms.

Also, functional material is extracted by inducing functionality microorganisms that are added microorganism extract after incubating bulk of strong proliferation in situ neutral microorganisms.

Also, various object material concentrated in nucleus, mitochondria, cytoplasm, vacuole, lysosome and various organelles in cell is extracted by homogenizing.

Also, after increasing activity of pest control enzyme including chitinase and 1,3-glucanase by heating for 20 minutes under pH3.2, an elicitor is extracted by incubated microorganisms.

Also, when incubating microorganisms, lots of secondary metabolite are extracted by adding appropriate amounts of elicitor.

Also, when incubating a combination of microorganisms, functional material are extracted after maximizing pest control inducing effect, antimicrobial spectrum and pest resistance gene.

Also, when incubating microorganisms, various object material are extracted by applying function of microorganisms that changes or modifies both raw material and functional mineral material including germanium, or animal and plant material including fir, cypress, licorice, and medicinal mushroom ,chemically.

Example of a technical feature of a present invention are given below:

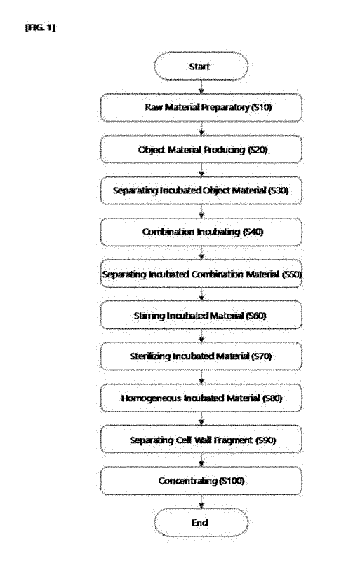

Referencing FIG. 1 that is a schematic configuration diagram showing a microorganism extract using the effective microorganisms and functionality microorganism and the production method, microorganism extract followed the present invention produce through producing methods including raw material preparatory stage (S10), object material producing stage (S20), separating incubated object material stage (S30) combination incubating stage (S40), separating incubated combination material stage (S50), stirring incubated combination material stage (S60), sterilizing incubated combination material stage (S70), microorganism homogeneous stage (S80), cell wall fragment separating stage (S90) and concentrating both incubated combination material and microorganism homogeneous material stage (S100).

Raw material preparatory stage (S10) includes choosing appropriate raw material for incubating microorganisms, measuring raw material using incubation, eliminating impurities and sterilizing raw material using ultra high temperature.

At this time, sterilizing is recommended using an autoclave with 6 kg/cm² of high temperature.

Object material producing stage (S20) includes inoculating anyone or any of several functionality microorganisms, anyone or any of several effective microorganisms in previous preparatory stage material and incubating 20° C.˜35° C., 35° C.˜60° C. for 3˜5 days, separately.

At this time, effective microorganisms is isolated in soil including Bacillus sp., lactic acid bacteria genus, Saccharomyces sp. Actinomyces sp. and photosynthesis bacteria genus and functionality microorganisms is isolated soil including Azotobacter sp., Pseudomonas sp., Aspergillus sp., Penicillium sp., Clostridium sp. and Trichoderma sp.

So, object material producing stage (S20) includes sole incubating and mix incubating under optimal microorganism growth condition for producing object material.

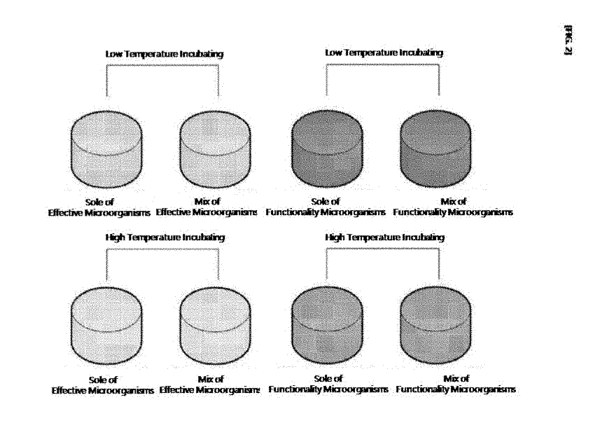

So, like a FIG. 2 that is a schematic configuration diagram showing an example of a culturing tank followed a microorganism extract using the effective microorganisms and functionality microorganism and the production method, incubating tanks are distinguished 20˜35° C. and 35˜60° C. each other and are also distinguished sole of effective microorganism/mix of effective microorganisms and sole of functionality microorganism/mix of functionality microorganisms each other.

35˜60° C. incubating tanks are also distinguished sole of effective microorganism/mix of effective microorganisms and sole of functionality microorganism/mix of functionality microorganisms each other.

Incubating sole and mix microorganisms, various object material are produced in each tanks.

Separating incubated object material stage (S30) separates raw material and object material in incubated each tanks through the object material producing stage (S20).

At this time, separating incubated object material in an incubating tank is recommended using a centrifugal force under 15˜25 kg/cm².

Combination incubating stage (S40) mixes object material in each tanks at separating incubated object material stage (S30) according to purpose and mixed object material produced by using various mixing ratio and combinations is incubated in each tanks for 5˜7 days with raw material.

This stage is necessary for producing a new functional material through the competition and symbiosis of microorganisms together, not for sole incubating.

So, a new object material is included in each combination tanks through the combination incubating with object material in each combination tanks.

Separating incubated combination material stage (S50) separates combination incubating material produced during previous combination incubating stage (S40) and raw material, separately

At this time, separating incubated combination material is recommended using a centrifugal force under 15˜25 kg/cm².

Stirring incubated combination material stage (S60) stirs each combination incubating material separated during separating incubated combination material stage (S50) according to purpose.

At this time, stirring incubated combination material stage (S60) is recommended stirring combination incubating material in each stirring tank with 200˜250 rpm/min in stirring tanks for 3 days.

Sterilizing incubated combination material stage (S70) sterilizes microorganisms in each combination incubating material stirred during previous stirring incubated combination material stage (S60) by using ultraviolet ray

At this time, sterilizing incubated combination material stage (S70) is recommended sterilizing combination incubating material by using 30,000 μW·lm/m³ ultraviolet ray

Because the conventional probiotics is produced alive microorganism itself for object material but present invention is produced object material already that is included optimal growth microorganism with combining material itself, sterilizing incubated combination material stage (S70) is a differentiated factor.

So, microorganisms through the sterilizing incubated combination material stage (S70) is sterilized.

Microorganism homogeneous stage (S80) homogenizes microorganism cell sterilized during previous sterilizing incubated combination material stage (S70) by using high pressure

At this time, microorganism homogeneous stage (S80) is recommended homogenizing microorganism cell by using 2,000 bar/cm² pressure.

Microorganism homogeneous stage (S80) extracts various object material in dead microorganisms concentrated in nucleus, mitochondria, cytoplasm, vacuole, lysosome and various organelles in cell that is extracted by homogenizing through sterilizing.

Cell wall fragment separating stage (S90) separates homogeneous material and homogeneous cell wall fragment made during previous microorganism homogeneous stage (S80).

At this time, cell wall fragment separating stage (S90) is recommended separating homogeneous material and homogeneous cell wall fragment by using 40˜50 kg/cm² of centrifugal force.

Concentrating both incubated combination material and microorganism homogeneous material stage (S100) concentrates homogeneous material without homogeneous cell wall fragment separated during cell wall fragment separating stage (S90).

At this time, concentrating both incubated combination material and microorganism homogeneous material stage (S100) is recommended concentrating homogeneous material with 1.2˜1.5 specific gravity under 60° C.

Like a followed explanation, microorganism extract is the present invention through the method of microorganism extract production.

So, The products of microorganism homogeneous extract that is extracting both incubated effective microorganisms and functionality microorganisms incubating sole and mixing each other because of producing lots of complex enzyme(proteins, sugars, fatty acids, cellulose, acid and alkali synthetic enzyme and decomposition enzyme etc.), primary metabolite, secondary metabolite, antibiotics, various active materials to form new microorganisms ecological systems including subtle and complex symbiotic relationship and microorganism inducer, plant growth promoter, plant pest resistance inducer etc. by homogenizing microorganisms, microorganism extract are sterilized and concentrated.

Example of an application of a present invention are given below:

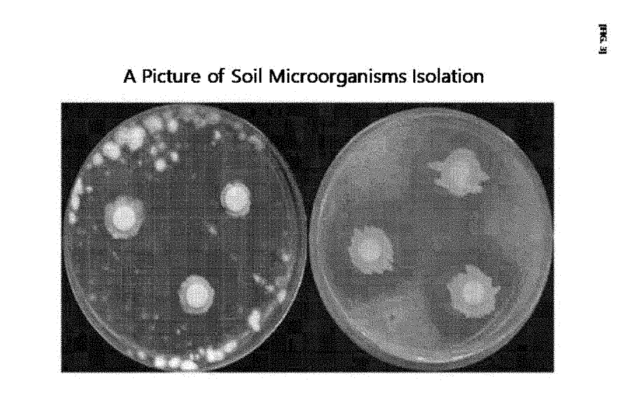

Like a FIG. 3 that a schematic configuration diagram showing a picture of soil microorganisms isolation followed a microorganism extract using the effective microorganisms and functionality microorganism and the production method, ecological, physical and biochemical feature is seized by selecting optimal species through the general method of identification of soil microorganisms.

It is studied that condition of optimal growth of selected optimal species, antibiotic activity material and growth promoting material in selected optimal species, that physical-chemical condition including temperature, hydrogen ion concentration and dissolved oxygen concentration and that limitation media, complex media and media condition including carbon, nitrogen, phosphoric acid, inorganic salt and cofactor for identification of optimal growth of selected optimal species, antibiotic activity material and growth activity in selected optimal species.

Activity of extracellular enzyme of isolated species is studied by using Bradford method(1976) for extracellular enzyme secretion that is used Bovine serum albumin for standard.

Researching and measuring safety through the field test for studying antagonistic microorganism availability is used pepper (Capsicum annum L.) seeds for target plant that is checked periodically for 120 days after transplanting seedling pepper until growing 5˜7 leaves.

For mass culturing, the selected optimal species incubates under 35° C., 100 rpm for 72 hours by using rich bran (5˜10%) as carbon sources.

Both complex enzyme, primary metabolite, secondary metabolite, antibiotics, various active materials etc. when incubating selected optimal species and microorganism inducer, plant growth promoter, plant pest resistance inducer etc. by homogenizing microorganisms are used.

At this time, growth about temperature and pH is confirmed to range of 20° C.˜50° C., pH 5.0˜8.0, optimal growth temperature is confirmed to 35° C. and optimal growth pH is confirmed pH 6.5 after decided growth about many kind of media at optimal growth condition of selected species by using many complex media(LB, YMG, NB and LME etc.) under pH 6.0.

Resulting of using limitation media for studying of effect of carbon sources, nitrogen sources and phosphate sources, optimal carbon source is maltose, optimal organic nitrogen source is yeast extract and optimal phosphate source is NH₄H₂PO₄.

Resulting of measuring growth state hourly for producing antibiotics and growth promoting material hourly, log phase is after 16 hours, stationary phase is after 72 hours, enzyme and metabolites are produced after 24 hours and producing of antibiotics, growth promoting material and various functional material is originated when producing primary and secondary metabolite.

Effect of microorganism extract followed present invention is confirmed by researching for growth of pepper and pest control.

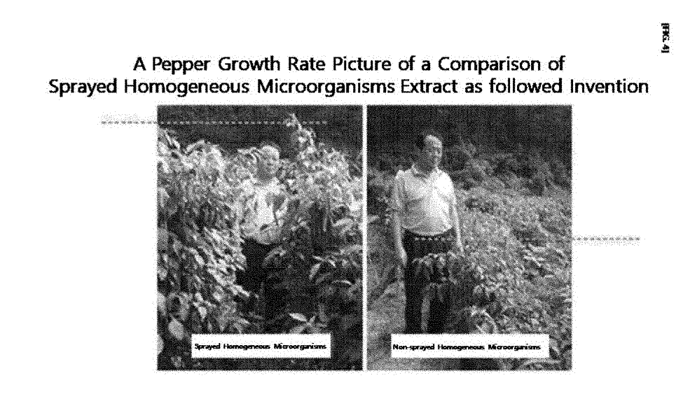

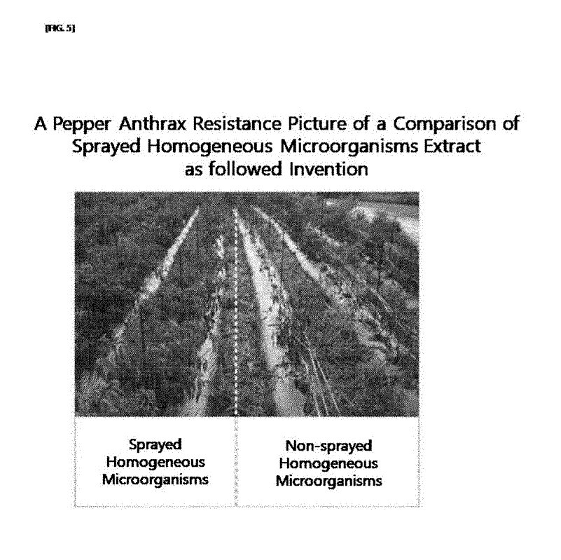

Like both FIG. 4 that is a schematic configuration diagram showing a pepper growth rate picture of a comparison of sprayed a microorganism extract using the effective microorganisms and functionality microorganism and non-sprayed and FIG. 5 that is a schematic configuration diagram showing a pepper anthrax resistance picture of a comparison of sprayed a microorganism extract using the effective microorganisms and functionality microorganism and non-sprayed, result of comparison test group A that is without Clostridium coccoides and test group B, C that is flow the Clostridium coccoides over surface leaves of young pepper grew until 5 leaves, test group A is showed normal growth, test group B is got wilting symptom to almost pepper and low yield and test group C is more lengthen stem, thicken fruit and showed high yield than test group A by spreading present invention on soil and surface of leaves after getting anthrax.

Especially, soil spraying at early stage of growth is showed a powerful significant result and like a TABLE 1, growth of treated test group using present invention is showed outstanding.

Also, after experiment, result of treatment to get wilt pepper, anthrax symptom is released.

This result, development the eco-friendly fertilizer and pesticide is considered through the complex enzyme, primary metabolite, secondary metabolite, antibiotics, various active materials, microorganism inducer, plant growth promoter and plant pest resistance inducer.

TABLE 1

| Experimental | Pepper fruit | | length | yield |
|---|---|---|---|---|
| group | Weight(g) | Size(cm) | Size(cm) | Count/stem |
| A | 17.5 ± 0.5 | 9.4 × 1.8(±1.0 × ±0.3) | 94 ± 10 | 17 ± 4 |
| B | 2.6 ± 0.8 | 6.2 × 1.0(±1.5 × ±0.3) | 68 ± 10 | 7 ± 4 |
| C | 20.5 ± 1.5 | 11.5 × 2.0(±1.0 × ±0.3) | 168 ± 10 | 27 ± 4 |

A: Control(microorganism extract: non-treated)
B: Pest spread(microorganism extract: non-treated)
C: Treated on soil and surface of leaves(microorganism extract: spread soil 1 time, surface of leaves 4 times)

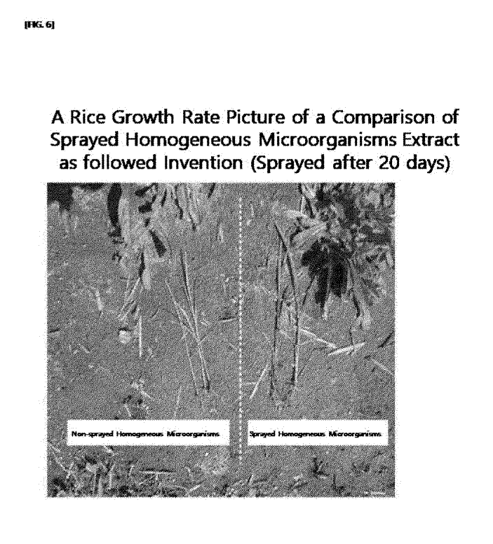

Additionally, like a FIG. 6 that is a schematic configuration diagram showing a rice growth rate picture of a comparison of sprayed a microorganism extract using the effective microorganisms and functionality microorganism and non-sprayed treated rice is more outstanding about growth of leaves, stem and roots than non-treated.

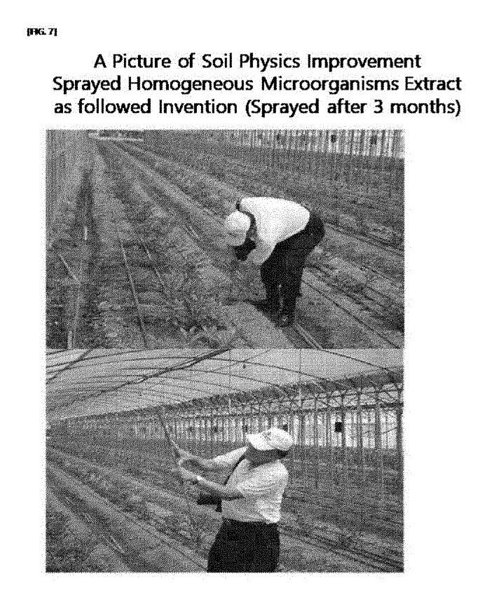

Also, like a FIG. 7 that is a schematic configuration diagram showing a picture of soil physics improvement sprayed a microorganism extract using the effective microorganisms and functionality microorganism after 3 months, spraying microorganism extract on soil surface is confirmed improvement of soil physics.

While the present invention has been described in connection with what is presently considered to be practical exemplary embodiments, it is to be understood that the invention is not limited to the disclosed embodiments, but, on the contrary, it will be obvious to those skilled in the art that such variations and modifications are within the scope of the appended claims.

What is claimed is:

1. Microorganism extract using effective microorganisms isolated in soil including Bacillus sp., lactic acid bacteria genus, Saccharomyces sp. Actinomyces sp. and photosynthesis bacteria genus and functionality microorganisms isolated soil including Azotobacter sp., Pseudomonas sp., Aspergillus sp., Penicillium sp., Clostridium sp. and Trichoderma sp. is produced complex enzyme and various inducer and microorganism extract by using effective microorganisms and functionality microorganisms isolated all in the soil for and the production method. The products of microorganism homogeneous extract that is extracting both incubated effective microorganisms and functionality microorganisms incubating sole and mixing each other because of producing lots of complex enzyme(proteins, sugars, fatty acids, cellulose, acid and alkali synthetic enzyme and decomposition enzyme etc.), primary metabolite, secondary metabolite, antibiotics, various active materials to form new microorganisms ecological systems including subtle and complex symbiotic relationship and microorganism inducer, plant growth promoter, plant pest resistance inducer etc. by homogenizing microorganisms, microorganism extract are sterilized and concentrated; is a feature.

2. Microorganism extract using the effective microorganisms and functionality microorganism producing method included following steps, preparatory stage including measuring raw material using incubation, eliminating impurities, sterilizing raw material using ultra high temperature; object material producing stage including inoculating anyone or any of several functionality microorganisms, anyone or any of several effective microorganisms in previous preparatory stage material and incubating 20° C.˜35° C., 35° C.˜60° C. for 3˜5 days, separately; separating stage including separating object material produced during previous object material producing stage and media in incubating tanks, separately; combination incubating stage including mixing object material separated previous separating stage depending on the blending ration according to purpose and incubating with culturing raw material for 5˜7 days, separately; separating stage including separating combination incubating material produced during previous combination incubating stage and raw material, separately; stirring stage including stirring each combination incubating material separated during previous separating stage according to purpose; sterilizing stage including sterilizing microorganisms in each combination incubating material stirred during previous stirring stage by using ultraviolet ray; microorganism homogeneous stage including homogenizing microorganism cell sterilized during previous sterilizing stage by using high pressure; cell wall fragment separating stage including separating homogeneous material and homogeneous cell wall fragment made during previous microorganism homogeneous stage; concentrating stage including concentrating homogeneous material without homogeneous cell wall fragment separated during cell wall fragment separating stage.

3. The method of claim 2, sterilizing stage including sterilizing combination incubating material by using 30,000 μW·lm/m³ ultraviolet ray; is a feature of microorganism extract using the effective microorganisms and functionality microorganism producing method.

4. The method of claim 2, microorganism homogeneous stage including homogenizing microorganism cell by using 2,000 bar/cm² of pressure; is a feature of microorganism extract using the effective microorganisms and functionality microorganism producing method.

5. The method of claim 2, cell wall fragment separating stage including separating homogeneous material and homogeneous cell wall fragment by using 40˜50 kg/cm² centrifugal force; is a technical feature of microorganism extract using the effective microorganisms and functionality microorganism producing method.

6. The method of claim 2, concentrating stage including concentrating homogeneous material with 1.2˜1.5 specific gravity under 60° C.; is a feature of microorganism extract using the effective microorganisms and functionality microorganism producing method.
